(19) United States
(12) Patent Application Publication
(10) Pub. No.: US 2005/0096466 A1
(43) Pub. Date: May 5, 2005

(54) Process for the preparation of tetrazol-derived compounds as growth hormone secretagogues

(76) Inventors: Mark D. Schwinden, Apex, NC (US); Zhongmin Xu, Princeton, NJ (US); Jollie Duaine Godfrey JR., Ewing, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/945,194

(22) Filed: Sep. 20, 2004

Related U.S. Application Data
(60) Provisional application No. 60/504,664, filed on Sep. 19, 2003.

(51) Int. Cl. C07D 201/02; C07D 207/267; C07D 211/44

(52) U.S. Cl. 540/200; 540/538; 546/218; 548/554

(57) ABSTRACT

A process for the preparation of tetrazole-derived compounds useful as growth hormone secretagogues is described.

### RELATED APPLICATIONS

This application claims priority benefit under Title 35 § 119(e) of U.S. provisional Application No. 60/504,664, filed Sep. 19, 2003, the contents of which are herein incorporated by reference.

### FIELD OF THE INVENTION

The present invention relates to processes for the preparation of organic molecules, specifically tetrazole-derived compounds, which are useful as growth hormone secretagogues.

### BACKGROUND OF THE INVENTION

The pituitary gland secretes growth hormone that stimulates growth in body tissue capable of growing and affects metabolic processes by increasing rate of protein synthesis and decreasing rate of carbohydrate synthesis in cells. Growth hormone also increases mobilization of free fatty acids and use of free fatty acids for energy.

A process for the preparation of tetrazole-derived compounds has previously been described in WO 01/60784 assigned to Bristol-Myers Squibb Co.

This route, however, is not optimal. First, the Mitsunobu step uses a very expensive and undesirable reagent TMSN₃. In addition, the purification of the Mistunobu product involves a tedious chromatography step that is unsuitable for large-scale synthesis. Moreover, the overall synthesis is not very efficient, since it is linear and involves many protection-deprotection steps. Furthermore, many of the intermediates in Scheme A are non-crystalline and difficult to purify in a commercially meaningful scale-up.

Accordingly, there is a need for improved processes for the preparation of tetrazole-derived compounds, especially for processes that improve upon the safety and economic feasibility of the processes known in the art.

### SUMMARY OF THE INVENTION

The present invention is directed to a process for preparing tetrazole-derived compounds that have activity as growth hormone secretagogues, specifically compounds of formula I: 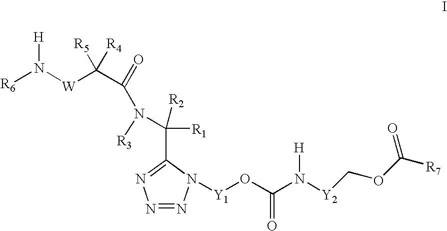
wherein
R₁ is selected from the group consisting of alkyl, cycloalkyl, alkenyl, cycloalkenyl, alkynyl, aryl and heterocycle;
R₂, R₃, R₄, R₅ and R₆ are each independently selected from the group consisting of hydrogen, alkyl, cycloalkyl and heterocycle, wherein R₄ and R₅ taken together may optionally form a cycloalkyl or heterocycle attached in a spiro fashion;
R₇ is selected from the group consisting of alkyl, cycloalkyl, alkenyl, cycloalkenyl, alkynyl, heterocycle, aryl, alkoxy and NR_{b}R_{c}, said R_{b} and R_{c} are each independently hydrogen, alkyl, cycloalkyl, heterocycle, aryl, or said R_{b} and R_{c} together with the N to which they are bonded optionally form a heterocycle;
W is a bond, —CH₂)ₚ—, or a cis- or trans- ethylene group —(CH═CH)ₚ;
Y₁ is —CH₂)ₙ;
Y₂ is —CH₂)ₘ;
p is an integer from 1 to 5;
n is an integer from 2 to 3;
m is an integer from 1 to 4;
   including all stereoisomers, prodrugs and pharmaceutically acceptable salts thereof; said process comprising the steps of:
   (a) reacting a compound of formula IV with a compound of formula VIII, wherein R₁, R₂, R₃, R₇, Y₁ and Y₂ are as defined above and G₁ is an amine protecting group, in the presence of an acid to produce a compound of formula V; 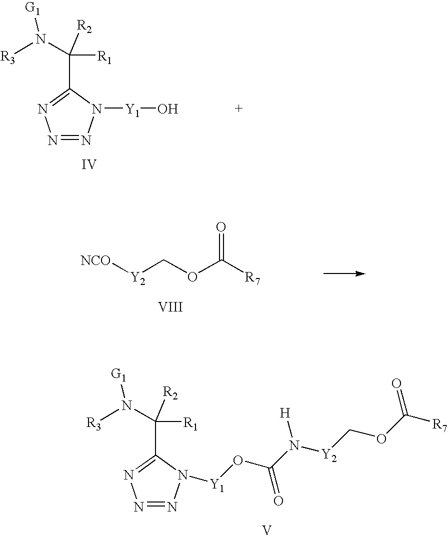
   (b) deprotecting the compound of formula V to form a compound of formula VI or a pharmaceutically acceptable salt thereof; 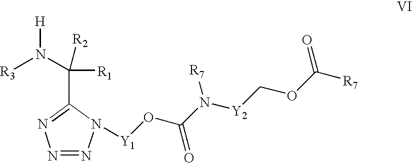
   (c) reacting a compound of formula VI or a pharmaceutically acceptable salt thereof with a compound of formula XI, in the presence of a peptide coupling reagent to give a compound of formula VII; and 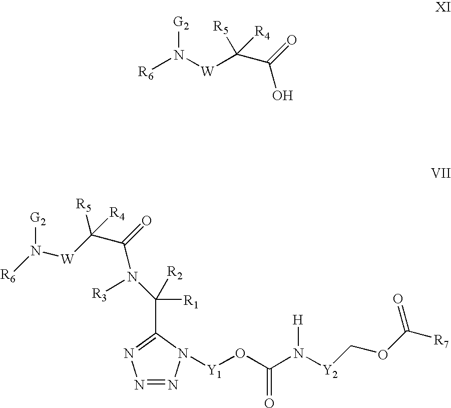
   (d) deprotecting the compound of formula VII.

A preferred process for making the compound of formula I, comprises the process wherein G₁ is selected from the group consisting of tert-butyloxycarbonyl, benzyloxycarbonyl and benzyl.

This invention is also directed to a process for making a compound of formula III: 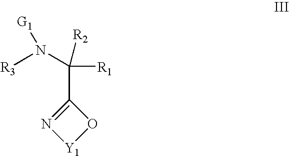
wherein
R₁ is selected from the group consisting of alkyl, cycloalkyl, alkenyl, cycloalkenyl, alkynyl, heterocycle and aryl;
R₂ and R₃ are each independently selected from the group consisting of hydrogen, alkyl, cycloalkyl and heterocycloalkyl;
G₁ is an amine protecting group;
Y₁ is —(CH₂)ₙ—, where n is an integer from 2 to 3;
   comprising reacting a compound of Formula I₁, 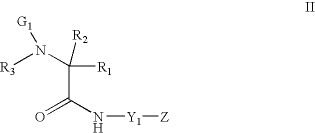
   wherein R₁, R₂, R₃, G₁ and Y₁ are as defined hereinabove, and Z is a leaving group selected from the group consisting of methanesulfonyloxy, p-toluenesulfonyloxy, chloro, bromo and iodo;
   with a suitable aqueous base in the presence of a phase transfer catalyst and an organic solvent.

A preferred process for making the compound of formula III, comprises the process wherein the aqueous base is NaOH or KOH.

Another preferred process for making the compound of formula III, comprises the process wherein the phase transfer catalyst is tetra n-butylammonium hydroxide, tetra n-butylammonium chloride, or Aliquat 336.

Another preferred process for making the compound of formula III, comprises the process wherein the organic solvent is selected from the group consisting of methylene chloride, chloroform, carbon tetrachloride, 1,2-dichloroethane, diethyl ether, THF, 1,4-dioxane, methyl t-butyl ether, dimethoxymethane and ethylene glycol dimethyl ether.

This invention is also directed to a process for making a compound of formula IV: 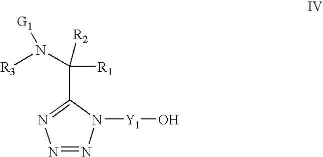
wherein
R₁ is selected from the group consisting of alkyl, cycloalkyl, alkenyl, cycloalkenyl, alkynyl, heterocycle and aryl;
R₂ and R₃ are each independently selected from the group consisting of hydrogen, alkyl, cycloalkyl and heterocycloalkyl;
G₁ is an amine protecting group; and
Y₁ is —CH₂)ₙ— where n is an integer from 2 to 3; comprising:
   (a) reacting a compound of formula III, 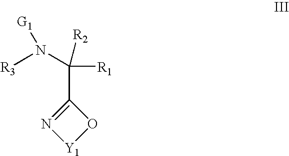
      wherein R₁, R₂, R₃, G₁ and Y₁ are as defined hereinabove, with a hydrazine or a salt of hydrazine in an alcohol solvent to produce a reaction mixture;
   (b) reacting the reaction mixture with a diazotization reagent in the presence of an acid.

A preferred process for making the compound of formula IV, comprises the process wherein the alcohol solvent is selected from the group consisting of MeOH, EtOH and isopropanol.

Another preferred process for making the compound of formula IV, comprises the process wherein the diazotization reagent is selected from the group consisting of sodium nitrite, iso-butyl nitrite and amyl nitrite.

Another preferred process for making the compound of formula IV, comprises the process wherein the acid is selected from the group consisting of HCl, methanesulfonic acid and phosphoric acid.

Another preferred process for making the compound of formula IV, comprises the process wherein G₁ is selected from the group consisting of tert-butyloxycarbonyl, benzyloxycarbonyl and benzyl.

### DETAILED DESCRIPTION OF THE INVENTION

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, isobutyl. The terms “alkyl” and “alk” includes substituted alkyl with one or more substituents, preferably 1 to 4 substituents, at any available point of attachment. Exemplary substituents include, but are not limited to, one or more of the following groups: hydrogen, halogen (e.g., a single halo substituent or multiple halo substituents forming, in the latter case, groups such as a perfluoroalkyl group or an alkyl group bearing Cl₃ or CF₃), trifluoromethyl, trifluoromethoxy, cyano, nitro, alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}Rₑ, C(═O)ORₑ, C(═O)Rₐ, C(═O)NR_{b}Rₑ, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is Rₐ except hydrogen. In the aforementioned exemplary substituents, groups such as alkyl, cycloalkyl, alkenyl, alkynyl, cycloalkenyl, heterocycle and aryl can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, the aforementioned cycloalkyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups includes ethenyl or allyl. The term “alkenyl” also includes substituted alkenyl with one or more substituents, preferably 1 to 4 substituents, at any available point of attachment. Exemplary substituents include, but are not limited to, alkyl or substituted alkyl, as well as those groups recited above as exemplary alkyl substituents.

The term “alkynyl” refers to a straight or branched chain hydrocarbon radical containing from 2 to 12 carbon atoms and at least one carbon to carbon triple bond. Exemplary such groups include ethynyl. The term alkynyl also includes substituted alkynyl with one or more substituents, preferably 1 to 4 substituents, at any available point of attachment. Exemplary substituents include, but are not limited to, alkyl or substituted alkyl, as well as those groups recited above as exemplary alkyl substituents.

The term “cycloalkyl” refers to a fully saturated cyclic hydrocarbon group containing from 1 to 4 rings and 3 to 8 carbons per ring. Exemplary such groups include cyclopropyl, cyclobutyl, cyclopentyl, cyclohexyl, cycloheptyl, etc. The term “cycloalkyl” also includes substituted cycloalkyl with one or more substituents, preferably 1 to 4 substituents, at any available point of attachment. Exemplary substituents include, but are not limited to, nitro, cyano, alkyl or substituted alkyl, as well as those groups recited above as exemplary alkyl substituents. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, the aforementioned cycloalkyl, cycloalkenyl, heterocycle and aryl can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. The term “cycloalkenyl” also includes substituted cycloalkenyl with one more substituents, preferably 1 to 4 substituents, at any available point of attachment. Exemplary substituents include but are not limited to nitro, cyano, alkyl or substituted alkyl, as well as those groups recited above as exemplary alkyl substituents. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, the aforementioned cycloalkyl, cycloalkenyl, heterocycle and aryl can themselves be optionally substituted.

The term “aryl” refers to cyclic, aromatic hydrocarbon groups which have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “aryl” also includes substituted aryl by one or more substituents, preferably 1 to 3 substituents, at any point of attachment. Exemplary substituents include, but are not limited to, nitro, cycloalkyl or substituted cycloalkyl, cycloalkenyl or substituted cycloalkenyl, cyano, alkyl or substituted alkyl, as well as those groups recited above as exemplary alkyl substituents. Exemplary substituents also include fused cyclic, especially fused cycloalkyl, fused cycloalkenyl, fused heterocycle, or fused aryl, the aforementioned cycloalkyl, cycloalkenyl, heterocycle and aryl can themselves be optionally substituted.

The terms “heterocycle,” “heterocyclic” and “heterocyclo” refer to fully saturated, or partially or fully unsaturated, including aromatic (i.e., “heteroaryl”) cyclic groups (for example, 4 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

The terms “heterocycle,” “heterocyclic” and “heterocyclo” include heterocycle, heterocyclic or heterocyclo groups substituted with one or more substituents, preferably 1 to 4 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl or substituted alkyl, as well as those groups recited above as exemplary alkyl substituents. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, the aforementioned cycloalkyl, cycloalkenyl, heterocycle and aryl can themselves be optionally substituted.

The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The terms “halogen” or “halo” refer to chlorine, bromine, fluorine or iodine.

The term “phase transfer catalyst” refers to a small quantity of a chemical agent that enhances the rate of a reaction between chemical species located in different phases (immiscible liquids or solid and liquid) by extracting one of the reactants, most commonly an anion, across the interface into the other phase so that reaction can proceed. These catalysts include quaternary ammonium or phosphonium salts (e.g. tetraalkylammonium salts, wherein alkyl can be same or different), or agents that complex inorganic cations (e.g. crown ethers or other cryptands). The catalyst cation is not consumed in the reaction although an anion exchange does occur.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1999).

Unless otherwise indicated, any heteroatom with unsatisfied valences is assumed to have hydrogen atoms sufficient to satisfy the valences.

The compounds of a particular formula may form salts which are also within the scope of this invention. Reference to a compound of the particular formula herein is understood to include reference to salts thereof, unless otherwise indicated. The term “salt(s)”, as employed herein, denotes acidic and/or basic salts formed with inorganic and/or organic acids and bases. In addition, when a compound of the formula contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula may be formed, for example, by reacting a compound of formula I with an amount of acid or base, such as an equivalent amount, in a medium such as one in which the salt precipitates or in an aqueous medium followed by lyophilization.

The compounds of a particular formula which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of a particular formula which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound which, upon administration to a subject, undergoes chemical conversion by metabolic or chemical processes to yield a compound of the formula I, or a salt and/or solvate thereof. Solvates of the compounds of formula I include, for example, hydrates.

Compounds of the formula I, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclo rings.

Throughout the specifications, groups and substituents thereof may be chosen to provide stable moieties and compounds.

### Methods of Preparation

The compounds of the present invention may be prepared by methods such as those illustrated in the following Schemes 1 to 5. The symbols used in the schemes are defined as above unless otherwise indicated. Solvents, temperatures, pressures, and other reaction conditions may readily be selected by one of ordinary skill in the art. Starting materials are commercially available or can be readily prepared by one of ordinary skill in the art.

A compound of formula III can be prepared according to Scheme 1. Amide coupling of starting material 1-1 with an amino alcohol yields intermediate 1-2, which can be further converted to an intermediate of formula II, wherein Z is a leaving group selected from the group consisting of methanesulfonyloxy, p-toluenesulfonyloxy, chloro, bromo and iodo, via a coupling reaction with methanesulfonyl chloride, or p-toluenesulfonyl chloride, or a halogenation reaction to displace the hydroxy group in 1-2 to a halogen group (chloro, bromo and iodo) in compound II. Finally, the compound of formula III can be obtained by treating the intermediate of formula II with a suitable aqueous base in the presence of a phase transfer catalyst and an organic solvent.

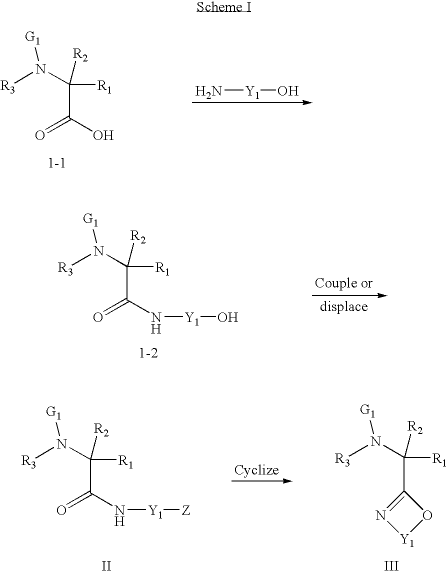

As illustrated in Scheme 2, a compound of formula IV can be prepared by reacting the compound of formula III with a hydrazine or a salt of hydrazine in an alcohol solvent to produce a reaction mixture, and then reacting the reaction mixture with a diazotization reagent in the presence of an acid.

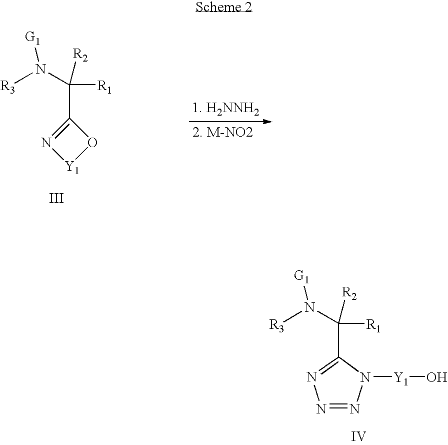

A compound of formula VI can be obtained according to Scheme 3, by treating the compound of formula IV with a isocyanate of formula VIII to afford a compound of formula V, followed by deprotection of the compound of formula V. Subsequent treatment of the compound of formula VI with an acid (A₁), for example, HCl, HBr, methylsulphonic acid, H3PO4, etc., yields a compound of formula VI-a. As shown in Scheme 3a, the isocyanate of formula VIII can be prepared by reacting a compound of formula 3-1 with a iodide salt (MI) such as NaI in the presence of an acid chloride of formula R₇C(═O)Cl, to yield a compound of formula 3-2. Treatment of compound 3-2 with an isocyanate salt (MOCN) such as KOCN in the presence of a tetrabutyl ammonium salt (Bu₄NX) such as tetrabutyl ammonium chloride, gives compound VIII.

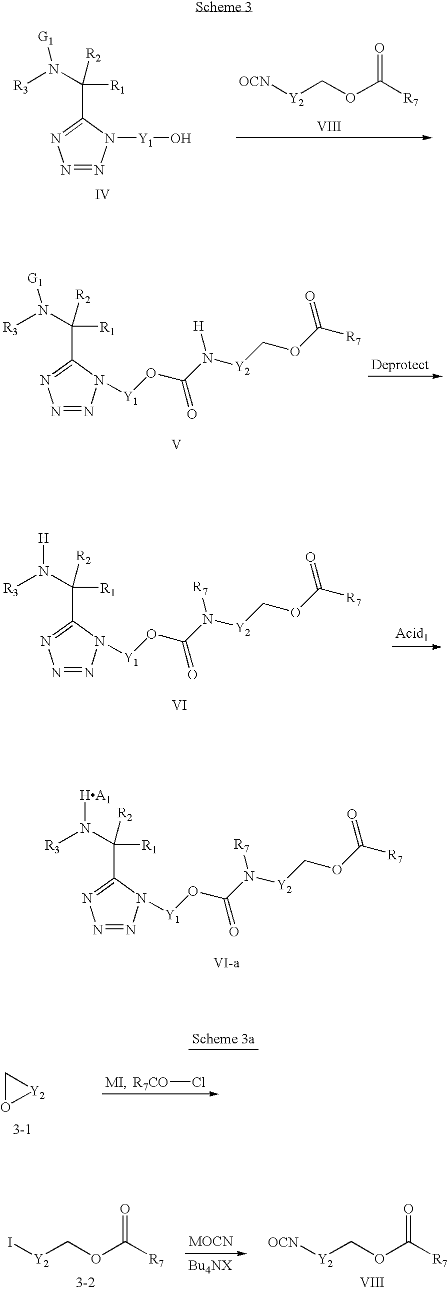

As illustrated in Scheme 4, the compound of formula I can be obtained via a coupling reaction of compound VI-a with an acid of formula XI, followed by deprotection. A salt form I-a can be obtained by treating compound I with an acid (A₂) such as HCl, H₃PO₄, etc.

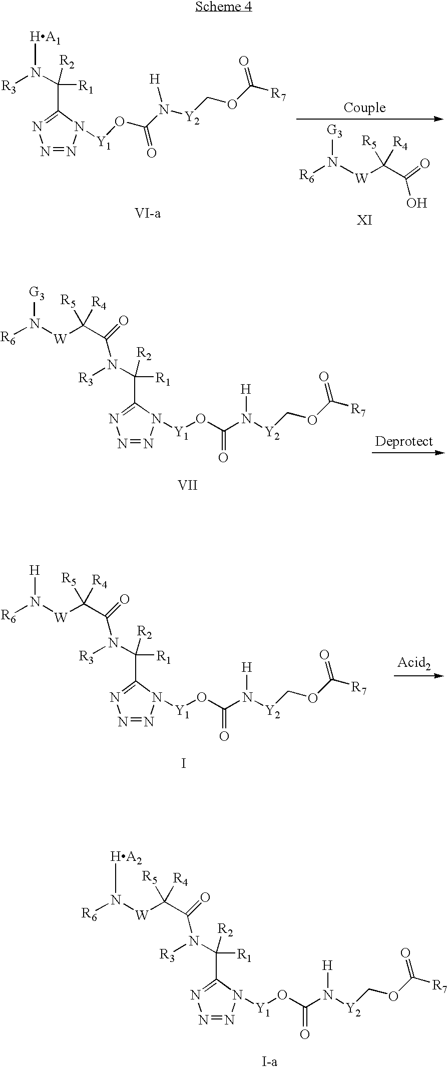

Alternatively, the compound of formula VI can be prepared according to Scheme 5. Treatment of compound 3-2 (see Scheme 3) with an isocyanate salt (MOCN) such as KOCN in the presence of a tetrabutyl ammonium salt (BU₄NX), for example, tetrabutyl ammonium chloride, gives compound VIII. Without isolation, compound VIII can be coupled with compound III (see Scheme 1) to give compound V, which can be deprotected in situ to give compound VI.

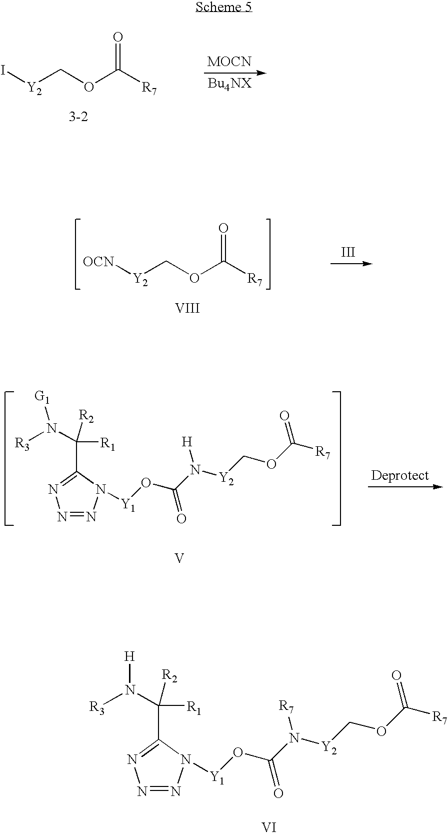

The present invention is further described by the following examples which are illustrative only, and are in no way intended to limit the scope of the instant claims. All references referred to in this specification are incorporated by reference in their entirety.

### Abbreviations

AcCl=acetyl chloride
AP=area percentage by HPLC
Boc=N-tert-butoxycarbonyl
bp=boiling point
BuOAC=n-butyl acetate
CSA=camphorsulfonic acid
DCM=dichloromethane
DMF=dimethyl formamide
DSC=differential scanning calorimetry
EtOAc=ethyl acetate
WSC=N′-(3-dimethylaminopropyl)-N-ethylcarbodiimide hydrochloride
HOBT=1-hydroxybenzotriazole
g=gram(s)
IBCF=isobutyl chloroformate
L=liter
KF=Karl Fischer
MeCN=acetonitrile
MeOH=methanol
NMM=n-methylmorpholine
MsCl=methanesulfonyl chloride
mg=milligram(s)
mL=milliliter
mmol=millimole
mol=mole
mp=melting point
MTBE=tert-butyl methyl ether
RT=room temperature
TEA=triethylamine

### EXAMPLES

### Example 1

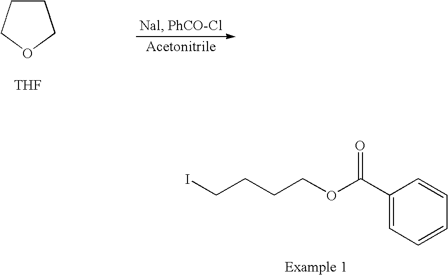

Into a solution of tetrahydrofuran (1.00 L, 12.33 mole) in acetonitrile (3.5 L), sodium iodide (2.21 kg, 14.735 mole) was added under a N₂ atmosphere. The suspension was cooled to 0-5° C., and a solution of benzoyl chloride (1.73 kg, 12.33 mole) in acetonitrile (0.37 L) was added slowly, keeping the temperature below 5° C. The resulting mixture was warmed to 23° C. and stirred at this temperature. After 24 h, the reaction mixture was diluted with MTBE (14.45 L), washed with 10% sodium hydrogen sulfite (2×10.84 L), saturated sodium bicarbonate solution (7.23 L), brine (7.23 L) and concentrated under vacuum to dryness to afford 3.71 kg of Example 1 as a pale yellow oil (99.3% yield, HPLC area percentage (AP)>99). ¹HNMR (CDCl₃, 400 mHz) δ 8.03 (d, J=8.3, 2H), 7.50 (m, 1H), 7.30 (d, J=7.7 Hz, 2H), 4.35 (t, J=6.5 Hz, 2H), 3.26 (t, J=6.8, 2H), 2.15-1.85 (m, 2H).

¹³CNMR (CDCl₃, 125 mHz) δ 166.5, 133.1, 130.3, 129.6, 128.5, 63.8, 30.2, 29.8, 6.1.

### Example 2

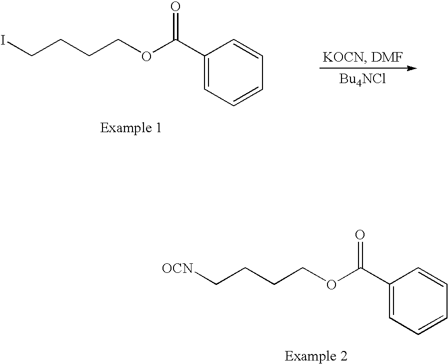

A mixture of tetrabutylammonium chloride (0.18 kg, 0.66 mole) and KOCN (0.80 kg, 9.86 mole) in 15 L of DMF was heated under vacuum at 60° C. to distill off 5.0 L of DMF, at which point, the KF value should be <0.02%. (If it is higher, add another 5.0 L of DMF and distill off another 5.0 L of DMF). Example 1 (1.0 kg, 3.29 mole) was added. The mixture was heated to 80° C. and stirred at this temperature for about 45 min. The mixture was then cooled to 0-5° C., diluted with cold MTBE (10.0 L, 4° C.), quickly washed with cold water (10.0 L, 4° C.), cold brine (10.0 L, 4° C.), dried over anhydrous sodium sulfate (0.80 kg), filtered and concentrated under vacuum below 35° C. to afford 0.62 kg of crude product as an brown oil with ˜70 AP. The crude product was purified by wiped-film evaporation (115-120° C./1×10⁻³ mbar) to give 0.35 kg of Example 2 (48% yield, AP>95) as a colorless oil, which solidifies when stored at <−20° C. ¹³CNMR, (CDCl₃) δ 166.5, 133.0, 130.1, 129.5, 128.3, 122.0, 64.0, 42.6, 27.9, 25.9.

¹HNMR (CDCl₃) d 8.04-8.03 (m, 2H), 7.58-7.55 (m, 1H), 7.46-7.43 (m, 2H), 4.37-4.34 (m, 2H), 3.42-3.39 (m, 2H), 1.91-1.84 (m, 2H), 1.81-1.75 (m, 2H).

### Example 3

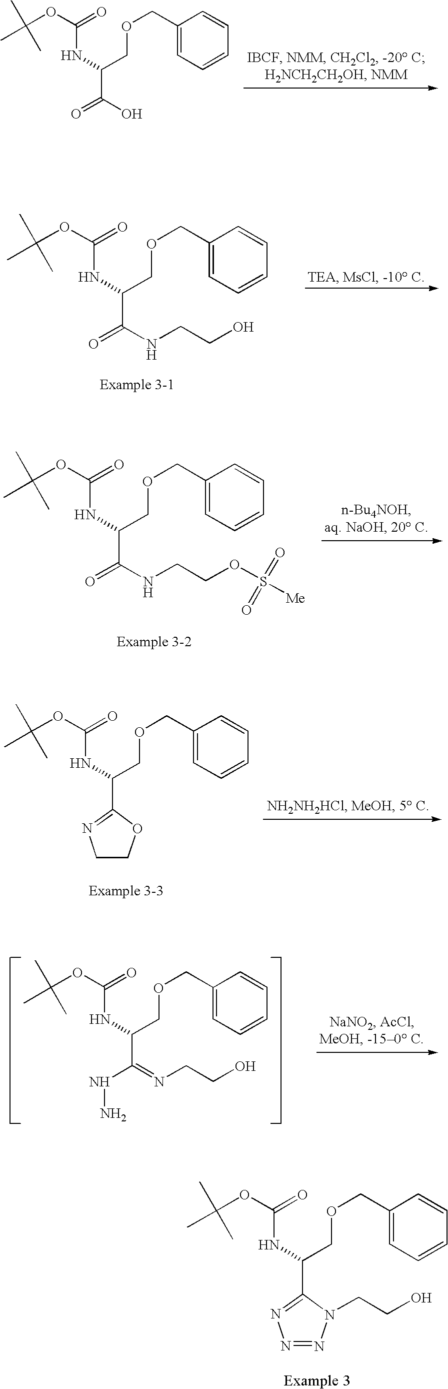

### Example 3-1

To a pre-cooled solution of N-Boc-O-benzyl-D-serine (2.50 kg, 8.47 mol, −40° C.) in 16.0 L CH₂Cl₂ were added N-methylmorpholine (NMM, 0.94 kg, 9.32 mol) and isobutyl chloroformate (1.27 kg, 9.31 mol) slowly in that order via addition funnel maintaining the internal temperature below −40° C. The mixture was then stirred at −40° C. for 2 h followed by slow addition of ethanolamine (0.569 kg 9.323 mol) and NMM (0.943 kg, 9.323 mol) via addition funnel, maintaining the internal temperature below −40° C. The mixture was then warmed to 22° C. over 0.5 h and stirred at that temperature for 0.5 h. The mixture was carried over to the next step without isolation.

(A fraction of the mixture was isolated for characterization purpose: ¹HNMR (CDCl₃) δ 7.33-7.28 (m, 5H), 6.91 (m, 1H), 5.51 (d, 1H), 4.56-4.48 (m, 2H), 4.29 (br, 1H), 3.86 (dd, J=9.32, 4.28 Hz, 1H), 3.65 (t, J=5.04 Hz, 2H), 3.60 (dd, J=9.57, 6.04 Hz, 1H), 3.39 (q, J=5.54 Hz, 2H), 3.08 (br, 1H), 1.44 (s, 9H); ¹³CNMR, (CDCl₃) δ 171.6, 156.1, 137.8, 128.9, 128.4, 128.2, 80.8, 73.8, 70.3, 62.0, 54.7, 42.7, 28.7; HRMS calcd for C₁₇H₂₆N₂O₅ 338.1942 (C₁₇H₂₇N₂O₅, 339.2021), found (M+H⁺) 339.1928; IR (Neat) 3314, 1655, 1530, 1366, 1250, 1167 cm⁻¹; and [α]²⁵_{D}−9.222 (c=1.075, MeOH).)

### Example 3-2

The above reaction mixture was then cooled to −10 to −15° C. TEA (2.40 kg, 23.71 mol) and MsCl (2.33 kg, 20.32 mol) were slowly added in that order, maintaining the internal temperature below −10° C. The resulting mixture was stirred at −10° C. for 30 min. (A fraction of the mixture was isolated for characterization purpose: ¹H NMR (400 MHz, CDCl₃) δ 7.38-7.27 (m, 6H), 6.92 (m, 1H), 5.38 (br, 1H), 4.58-4.50 (m, 2H), 4.27 (t, J=5.04 Hz, 3H), 3.92 (dd, J=9.07, 3.53 Hz, 1H), 3.60 (m, 3H), 2.92 (s, 3H), 1.45 (s, 9H); ¹³CNMR, (CDCl₃) δ 170.9, 155.6, 137.4, 128.6, 128.0, 127.8, 80.5, 73.4, 69.8, 68.3, 54.1, 38.0, 37.2, 28.3; HRMS calcd for C₁₈H₂₈N₂O₇S 416.1617, found [M+H]⁺ 417.1693; IR (KBr) 3336, 1680, 1661, 1554, 1525, 1350, 1307, 1247, 1186, 1174, 1108, 920 cm⁻¹; and [α]²⁵_{D}−1.766 (c=1.086, MeOH).)

### Example 3-3

40% tetrabutyl ammonium hydroxide (0.27 kg, 0.42 mol) and 2.3 M NaOH (20.0 L, 46.60 mol) were added to the above reaction mixture. The resulting mixture was warmed to 22° C. and stirred for 14 h at this temperature. The stirring was stopped to allow the phases to separate. The organic layer was washed with 5% NaHCO₃ (28.0 L) and deionized water (20.0 L), and concentrated at 35-40° C. in vacuum (29 mmHg) to a total weight of 15.40 Kg with AP>93.

¹³CNMR, (100 MHz, CDCl₃) δ 166.1, 155.2, 137.8, 128.3, 127.6, 127.4, 79.7, 73.0, 69.8, 68.2, 54.2, 49.3, 28.4.

### Example 3

A solution of crude Example 3-3 (15.40 kg, ˜8.47 mole) in anhydrous methanol (1.96 kg) was added to a cold slurry of NH₂NH₂.HCl (0° C., 1.16 kg, 16.93 mol) in methanol (32.0 L) while maintaining the internal temperature below 5° C. The mixture was stirred at 0-5° C. for 1 h followed by adding sodium nitrite (0.73 kg, 10.62 mole), and then was cooled to −15° C. In a separate vessel a solution of anhydrous HCl was prepared by adding AcCl (1.13 kg, 14.42 mol) to methanol (4.0 L) at 0-5° C. The resulting HCl solution was added slowly to the reaction mixture via additional funnel while maintaining the internal temperature below 0° C. The addition was exothermic and the temperature rose to −1° C. After stirring at −10 to −5° C. for 40 min, the mixture was diluted with toluene (36.0 L) and then concentrated at 35° C. under vacuum (29 mmHg) until 38.0 L of organic were collected. The concentrated mixture was washed with saturated 5% NaHCO₃ solution (30.0 L) and deionized water (30.0 L), and concentrated at 40° C. in vacuum (29 mm Hg) to a weight of 15.4 kg. The concentrated solution was then heated to 50° C. followed by addition of n-heptane (7.67 kg) and seed crystals. The resulting slurry was allowed to cool to 22° C. over 6 h and stirred for 6 h at this temperature. The solid was filtered through an 18 inch polypropylene buchner funnel, washed with n-heptane (2×4.0 L) and dried in a vacuum oven (35° C., >29 mmHg) for 48 h to afford 1.85 kg of Example 3 as a white solid (62% yield, 100% ee and AP>98).

¹H NMR (400 MHZ, CDCl₃) δ 7.35-7.29 (m, 3H), 7.18 (d, J=8.1, 2H), 5.46 (d, J=8.01 Hz, 1H), 5.37 (dt, J=6.1, 8.2 Hz, 1H), 4.57 (m, 2H), 4.47 (d, J=11.4 Hz, 2H), 4.86-4.05 (m, 3H), 3.76 (t, J=8.0 Hz, 1H), 3.05 (t, J=6.6 Hz, 1H), 1.40 (s, 9H).

¹³C NMR (100 mHz, CDCl₃) δ 128.3, 127.9, 127.6, 136.9, 155.1, 155.4, 80.8, 73.5, 70.4, 60.7, 50.3, 44.9, 28.1.

HRMS calcd for C₁₇H₂₅N₅O₄ 416.1617, found [M+H] 364.1964.

Anal. Calcd. For C₁₇H₂₅N₅O₄: C, 56.26; H, 6.89; N, 18.97. Found: C, 56.18; H, 6.93; N, 19.27. [a]²⁵_{D}=+16.751° (c=1.004, MeOH).

### EXAMPLE 4

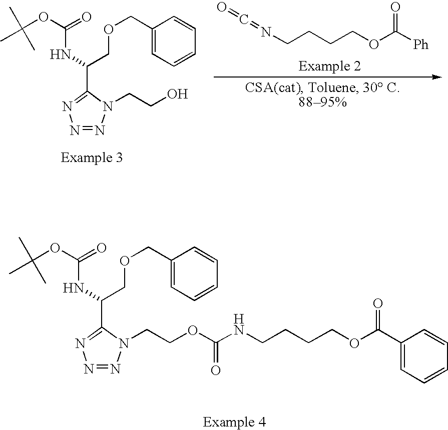

A mixture of Example 3 (1.44 kg, 3.96 mole), isocyanate Example 2 (1.00 kg, 4.56 mole) and racemic camphorsulfonic acid (0.14 kg, 0.20 mole) in 12.4 L of anhydrous toluene (KF<0.02%) was stirred at 23° C. for 4 h. 1.0 g of seed crystals was then added, and the resulting slurry was stirred at 20° C. for 18 h followed by adding n-heptane (6.2 L). The mixture was stirred at 20° C. for an additional 2 h, subsequently cooled to 0-5° C. and stirred at this temperature for 1 h. The solid was filtered, washed with a cold 2:1 toluene/n-heptane (4.3 L, 40° C.) and n-heptane (2.0 L), and air-dried under vacuum at 40° C. until a constant weight was obtained (2.30 kg, 3.95 mole, 100% yield, AP 95). The product was further purified as follows: the material was suspended in isopropanol (11.5 L) at 20-25° C., and the slurry was cooled to 0-5° C. and stirred at this temperature for 4 h. The solid was filtered, washed with cold isopropanol (4° C., 2×2.3 L) and dried under vacuum at 40° C. until a constant weight of 2.013 kg (87.5% recovery and >99 AP) was obtained. HRMS calcd for C₂₉H₃₈N₆O₄, found [M+H] 583.2841.

Anal. Calcd. C, 59.78; H, 6.57; N, 14.42. For C₂₉H₃₈N₆O₇: Found: C, 59.81; H, 6.47; N, 14.41. [a]²⁵_{D}=+4.713° (c=1.005, MeOH).

### Example 5

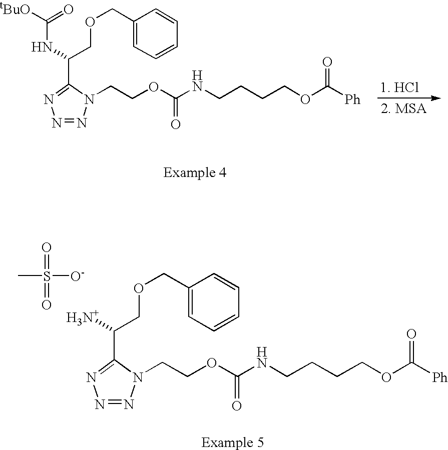

Gaseous HCl (0.50 kg, 13.70 mole) was bubbled through ethyl acetate (5.0 L) at 5° C. followed by the addition Example 4 (1.0 kg, 1.72 mole). The mixture was warmed to 20-25° C., stirred at this temperature for 1.5 h and then cooled to 0° C. 2 M NaOH solution (5.7 L) was added to adjust the pH to 9.0-9.5, while keeping the internal temperature below 20° C. The layers were separated, and the aqueous layer was washed with ethyl acetate (3.33 L). The combined organic layers were washed with water (4.0 L), brine (4.0 L) and partially concentrated under vacuum to a volume of 6.67 L. Into this concentrated solution, methanesulfonic acid (0.16 L, 2.47 mole), MTBE (2.0 L) and seed crystals were added in that order. The mixture was stirred at 20-25° C. for 3 h, cooled to 0-5° C. over 1 h and stirred for 1 h at this temperature. The solid was filtered, washed with a cold 2:1 EtOAc/MTBE (4° C., 1.0 L) and dried under vacuum at 40° C. to constant weight. The product was obtained as a white solid (0.83 kg, 1.312 mole, 76% yield). Anal. Calcd. For C₂₅H₃₄N₆O₈S: C, 51.89; H, 5.92; N, 14.52; S, 5.54. Found: C, 51.97; H, 5.96; N, 14.59; S, 5.54. MP=118.5-120.5° C.

[α]²⁵_{D}=+7.870 (c=1.0, 95% EtOH).
Alternative one-pot synthesis of Example 5:

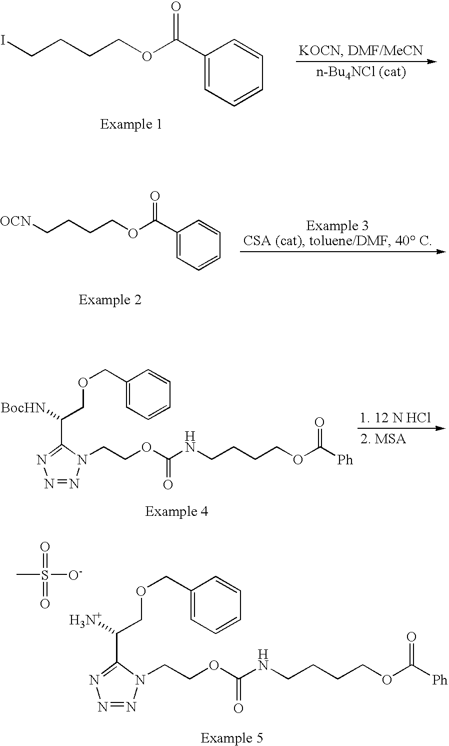

### Example 2

A slurry of KOCN (32.0 g, 3.94 mmol) and tetrabutylammonium iodide (15.0 g, 40 mmol) in 1.14 L of 9:1 of MeCN/DMF was heated to 86° C. to distill off ˜580 mL MeCN under vacuum over 4 h. At this point, the KF value should be <0.02%. (If it is higher, add another 580 mL of MeCN and distill off another 500 mL of MeCN). Example 1 (60 g, 197.3 mmol) was then charged, after which the internal temperature dropped to 81° C. The mixture was vigorously stirred for 16 hours at 80° C. and subsequently cooled to 50° C. 490 mL of toluene was then charged, and 680 mL MeCN was removed at 50° C. under vacuum to result in a 6:1 toluene/DMF solution containing ˜39 mmol of Example 2 (udged by HPLC analysis).

### Example 4

A slurry of Example 3 (46.0 g, 127 mmol) and camphorsulfonic acid (5.50 g, 19 mmol) in 200 mL of toluene was added after the above solution was cooled to 40° C. The resulting mixture was stirred at 40° C. for 6 h.

### Example 5

450 mL of toluene was removed at 50° C. under vacuum over 4 h. 158 mL of concentrated hydrochloric acid was added over 30 min followed by addition 450 mL of EtOAc. The resulting biphasic solution was vigorously stirred at 50° C. for 8 h. The reaction mixture was subsequent cooled to 0° C., and ˜320 mL of 6 M NaOH solution was added to adjust the pH to 12. The layers were separated and the organic layer was washed 1:1H₂O/brine (500 mL). The organic solution was concentrated at 50° C. under partial vacuum to distill off solvents (560 mL) and then diluted with 520 mL EtOAc. The resulting solution was polish filtered, and methanesulfonic acid (9.06 mL, 13.97 mmol) was added at 45° C. Seed crystals of Example 5 (100 mg) were added, and crystallization commenced. The solution was cooled to 22° C. over 5 h and stirred for an additional 8 h at this temperature. The crystals were filtered and washed with cold EtOAc (50 mL, 4° C.) and n-heptane (200 mL), and dried under vacuum to afford 55.7 g (97 mmol, 78% yield and >99 AP) as a white solid.

### Example 6

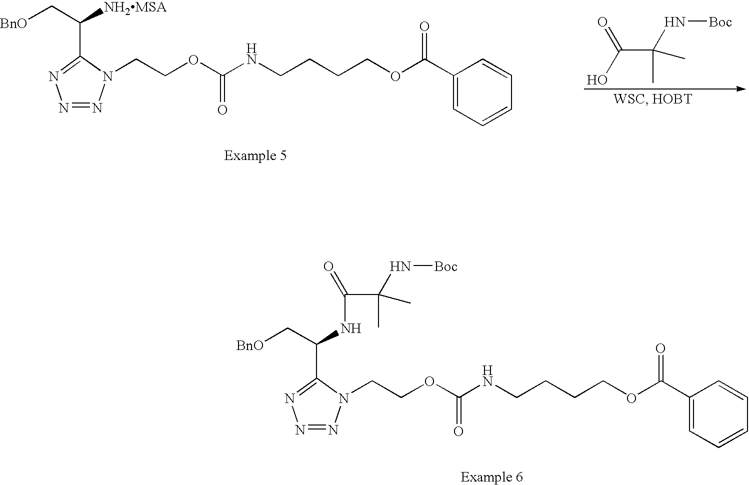

Into a clear solution of Example 5 (1.0 kg) in 10.0 L of dichloromethane was added diisopropylethylamine (0.58 kg), 1-hydroxy-1H-benzotriazole hydrate (HOBT, 0.26 kg), N′-(3-dimethylaminopropyl)-N-ethylcarbodiimide hydrochloride(WSC, 0.36 kg) and BOC-aminoisobutyric acid (0.39 kg) in that order. The mixture was then stirred at 20-25° C. for 12 h. The reaction mixture was then washed with water (2×6.0 L), 1 M HCl (2×5.0 L), water (6.0 L), saturated sodium bicarbonate solution (2×5.0 L) and concentrated under vacuum at −40° C. to afford 1.10 kg (95% yield) of product as a clear and almost colorless oil.

### Example 7

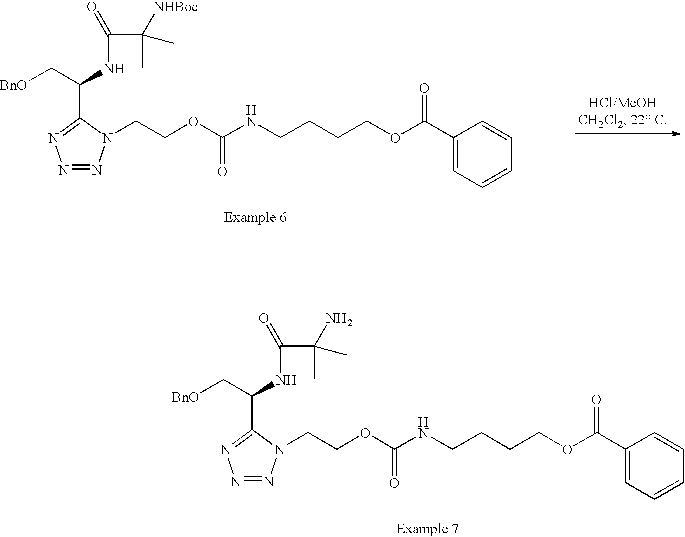

A clear solution of 1.0 kg of Example 6 in 10 L of dichloromethane was cooled to 0° C. 1.87 L of 4 M hydrochloric acid solution in methanol was then slowly added, keeping the internal temperature below 5° C. The mixture was allowed to warm to 20-25° C. and then stirred at this temperature for 40-48 h, after which complete consumption of the starting material was observed. The mixture was then cooled to ˜15° C. Water (10.0 L) and 4 M sodium hydroxide solution (1.46 L) were added to adjust the pH to 6.0-6.2. The solvents were then removed in vacuum, after which an aqueous emulsion with an oil was formed, and the pH was ˜7.5. The concentrated mixture was diluted with MTBE (10.0 L) and then cooled to 20° C. 1 M hydrochloric acid (1.63 L) was added to adjust the pH to ˜2.0. The layers were separated, and the aqueous layer was washed with MTBE (5.0 L) and ethyl acetate (10.0 L). The pH of the aqueous layer was adjusted to 7.0-7.5 from 2.0 by addition of 4 M sodium hydroxide solution (0.41 L). The layers were separated, and the aqueous phase was washed with ethyl acetate (5.0 L). The combined organic layers were washed with brine (5.0 L) and concentrated under vacuum at ˜40° C. to afford 0.78 kg (1.37 mole, 92% yield) of slightly colored oil which contained some NaCl. The oil was dissolved in ethyl acetate (7.0 L), and the mixture was filtered, and the solids were washed with ethanol (0.78 L). The filtrate was concentrated in vacuum at ˜40° C. to afford 0.76 kg (1.34 mole, 89.5% yield) of product as a clear and slightly colored oil.

¹H NMR (500 MHz, CDCl₃) δ 8.79 (d, J=9.8 Hz, 1H), 8.02 (d, J=8.8 Hz, 2H), 7.55 (t, J=8.8 Hz, 1H), 7.43 (t, J=7.7 Hz, 2H), 7.26-7.33 (m, 3H), 7.12 (d, J=7.7 Hz, 2H), 6.51 (t, J=5.5 Hz, 1H), 5.88 (dt, J=5.5, 9.3 Hz, 1H), 4.64-4.73 (m, 2H), 4.40-4.47 (m, 3H), 4.31 (t, J=6.6 Hz, 2H), 4.18 (dt, J=3.8, 9.3 Hz, 1H), 3.81 (dd, J=5.4, 8.2 Hz, 1H), 3.62 (t, J=9.3 Hz, 1H), 3.12-3.22 (m, 2H), 1.77-1.83 (m, 2H), 1.60-1.66 (m, 2H), 1.32 (s, 3H), 1.28 (s, 3H).

³C NMR (125 MHz, CDCl₃) δ 178.0, 166.4, 155.4, 136.7, 132.7, 130.2, 129.4, 128.4, 128.2, 128.0, 127.5, 73.4, 71.3, 64.4, 62.7, 54.7, 46.8, 43.2, 40.4, 28.9, 28.7, 26.2, 25.9.

IR (thin film) 3312, 1718, 1664, 1276 cm⁻¹.

HRMS: Anal. Calcd for C₂₈H₃₇N₇O₆, Found m/z 568.2885 (M+H⁺).

Anal. Calcd for C₂₈H₃₇N₇0600.35H₂O: C, 58.60; H, 6.62; N, 17.09. Found: C, 58.58; H, 6.39; N, 16.97.

[α]²⁵_{D}=−6.0°(c 1.0, MeCN).

### Example 8

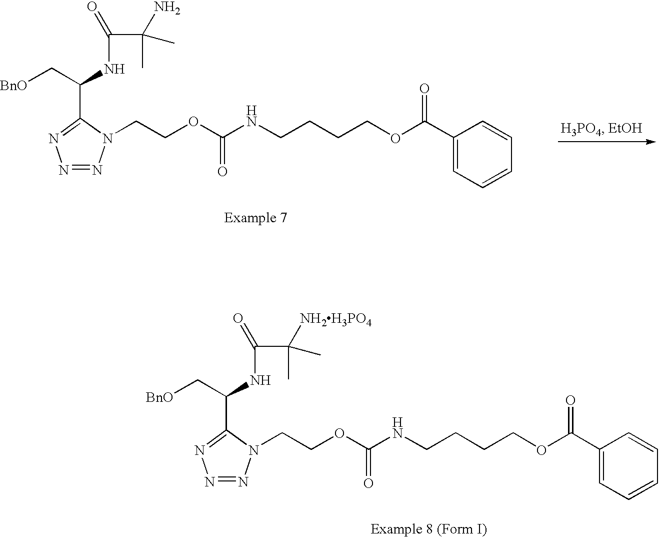

Under an inert gas atmosphere, 1.0 kg of Example 7 was dissolved in 29.0 L of ethanol at 40-45° C. The resulting solution was then heated to 72-74° C. Phosphoric acid (85%, 0.103 kg) was added over 5-10 min. The solution was cooled to about 60° C., seed crystals of form I were added. The solution became turbid and was cooled further to 53-55° C., and more seeds were charged. When the solid salt began to precipitate, the slurry was warmed to ˜65° C., and additional phosphoric acid (85%, 0.052 kg) was charged over 2-5 min. A thick suspension was observed. After 25 min more phosphoric acid (85%, 0.051 kg) was charged over 2-5 minutes. The suspension got thicker and was stirred at 65° C. for 10-20 min. The suspension was cooled to 20-25° C. over 4-5 hr, to 0-5° C. over 30-60 minutes and stirred at 0-5° C. for 1 h. The crystals were filtered, washed with cold ethanol (5.6 L, 4° C.) and dried under vacuum at 50-55° C. to constant weight. 1.03 kg (91% yield) of product was obtained as white crystals.

### Example 9

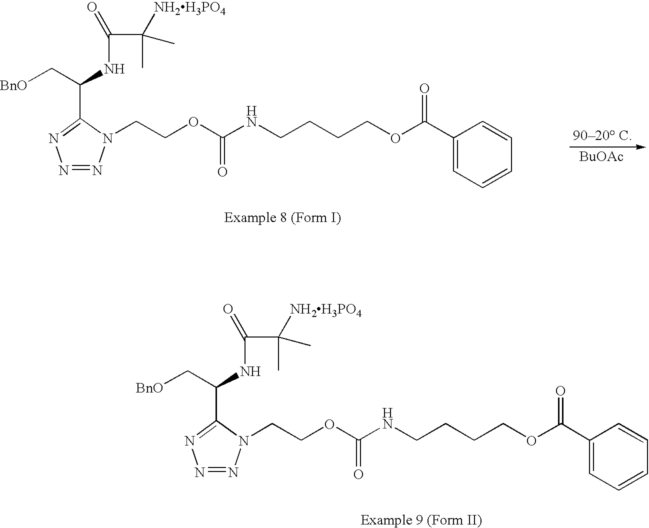

Under an argon atmosphere, a suspension of Example 8 (1.0 kg) in 20.0 L of butyl acetate was heated to 90° C. to obtain a slightly turbid and opalescent solution. After adding seeds of form II (0.5% input), the mixture was stirred at 90° C. for 1 h. Additional seed crystals of form 11 (0.5% input) were added, and the mixture was then cooled to 83-84° C. within 1 h and stirred at this temperature until the conversion to form II is complete. During this transformation an unstirrable slurry was observed and lasted for 10-30 minutes. After the conversion is complete, the suspension became thinner again and is again easily stirrable. The conversion of form I to form II crystals was monitored by DSC and took 2-4 h. The slurry was cooled over 4-5 h to 20-25° C. and stirred for 2 hr. The crystals were filtered, washed with butyl acetate (2×2.5 L) and dried under vacuum at 40-45° C. to constant weight. Example 9 (Form II) (0.95 kg, 95% yield) was obtained as white crystals.

¹H NMR (500 MHz, DMSO-d₆) δ 7.96 (d, 2H, J=6.6 Hz), 7.66 (t, 1H, J=7.4 Hz), 7.53 (t, 2H, J=7.7 Hz), 7.30-7.35 (m, 2H), 7.24-7.30 (m, 6H), 5.51 (t, 1H, J=6.6 Hz), 4.65-4.75 (m, 2H), 4.30-4.20 (m, 2H), 4.25 (t, 2H, J=6.6 Hz), 3.95-4.00 (m, 1H), 3.88-3.95 (m, 1H), 3.01 (q, 2H, J=6.3 Hz), 1.64-1.71 (m, 2H), 1.47-1.54 (m, 2H), 1.36 (s, 3H), 1.35 (s, 3H).

¹³C NMR (500 MHz, DMSO-d₆) δ 174.7, 166.0, 155.6, 154.4, 138.0, 133.5, 130.1, 129.3, 129.0, 128.5, 127.8, 127.7, 72.5, 69.5, 64.6, 61.7, 55.7, 46.9, 43.6, 26.1, 25.7, 25.5, 25.3.

[α]²⁵_{D} +8.280 (c=1.0, H₂O).

Anal. Calcd for C₂₈H₀N₇O₁₀P: C, 50.52; H, 6.06; N, 14.73; P, 4.65. Found: C, 50.39; H, 5.86; N, 14.65; P, 4.50.

IR (KBr) 2962, 1722, 1692, 1678, 1262, 1120, 1054 cm⁻¹.

1. A process for preparing a compound of formula III: 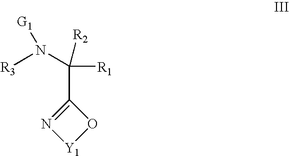
wherein
R₁ is selected from the group consisting of alkyl, cycloalkyl, alkenyl, cycloalkenyl, alkynyl, heterocycle and aryl;
R₂ and R₃ are each independently selected from the group consisting of hydrogen, alkyl, cycloalkyl and heterocycloalkyl;
G₁ is an amine protecting group;
Y₁ is —(CH₂)ₙ— where n is an integer from 2 to 3;
comprising reacting a compound of formula II, 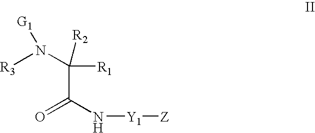
wherein R₁, R₂, R₃, G₁ and Y₁ are as defined hereinabove, and Z is a leaving group selected from the group consisting of methanesulfonyloxy, p-toluenesulfonyloxy, chloro, bromo and iodo;
with a suitable aqueous base in the presence of a phase transfer catalyst and an organic solvent.

2. The process of claim 1, wherein G₁ is an amine protecting group selected from the group consisting of tert-butyloxycarbonyl, benzyloxycarbonyl and benzyl.

3. The process of claim 1, wherein the aqueous base is LiOH, NaOH or KOH.

4. The process of claim 1, wherein the phase transfer catalyst is selected from the group consisting of tetra n-butylammonium hydroxide, tetra n-butylammonium chloride and Aliquat 336.

5. The process of claim 1, wherein the organic solvent is selected from the group consisting of: (a) a halogen-containing solvent selected from methylene chloride, chloroform, carbon tetrachloride and 1,2-dichloroethane and (b) an ether solvent selected from diethyl ether, THF, 1,4-dioxane, methyl t-butyl ether, dimethoxymethane and ethylene glycol dimethyl ether.

6. A process for preparing a compound of formula IV: 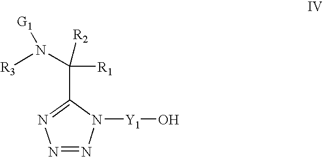
wherein
R₁ is selected from the group consisting of alkyl, cycloalkyl, alkenyl, cycloalkenyl, alkynyl, heterocycle and aryl;
R₂ and R₃ are each independently selected from the group consisting of hydrogen, alkyl, cycloalkyl and heterocycloalkyl;
G₁ is an amine protecting group; and
Y₁ is —(CH₂)ₙ— where n is an integer from 2 to 3;
comprising:
(a) reacting a compound of formula III, 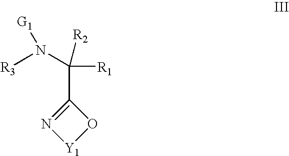
wherein R₁, R₂, R₃, G₁ and Y₁ are as defined hereinabove, with a hydrazine or a salt of hydrazine in an alcohol solvent to produce a reaction mixture; and
(b) reacting the reaction mixture with a diazotization reagent in the presence of an acid.

7. The process of claim 6, wherein the alcohol solvent is selected from the group consisting of MeOH, EtOH and isopropanol.

8. The process of claim 6, wherein the diazotization reagent is selected from the group consisting of sodium nitrite, iso-butyl nitrite and amyl nitrite.

9. The process of claim 6, wherein the acid is selected from the group consisting of HCl, methanesulfonic acid and phosphoric acid.

10. The process of claim 6, wherein G₁ is an amine protecting group selected from the group consisting of tert-butyloxycarbonyl, benzyloxycarbonyl and benzyl.

11. A process for preparing a compound of formula I: 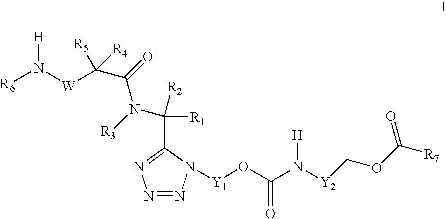
wherein
R₁ is selected from the group consisting of alkyl, cycloalkyl, alkenyl, cycloalkenyl, alkynyl, heterocycle and aryl;
R₂, R₃, R₄, R₅ and R₆ are each independently selected from the group consisting of hydrogen, alkyl, cycloalkyl and heterocycloalkyl, wherein R₄ and R₅ taken together may optionally form a cycloalkyl or heterocycloalkyl attached in a spiro fashion;
R₇ is selected from the group consisting of alkyl, cycloalkyl, alkenyl, cycloalkenyl, alkynyl, heterocycle, aryl, alkoxy and substituted amino;
W is a bond, —CH₂)ₚ—, or a cis- or trans- ethylene group —(CH═CH)ₚ;
Y₁ is —CH₂)ₙ;
Y₂ is —CH₂)ₘ;
p is an integer from 1 to 5;
n is an integer from 2 to 3; and
m is an integer from 1 to 4;
including all stereoisomers, prodrugs and pharmaceutically acceptable salts thereof;
said process comprising the steps of:
(a) reacting a compound of formula IV with a compound of formula VIII, wherein R₁, R₂, R₃, R₇, Y₁ and Y₂ are as defined above and G₁ is an amine protecting group, in the presence of an acid to produce a compound of formula V; 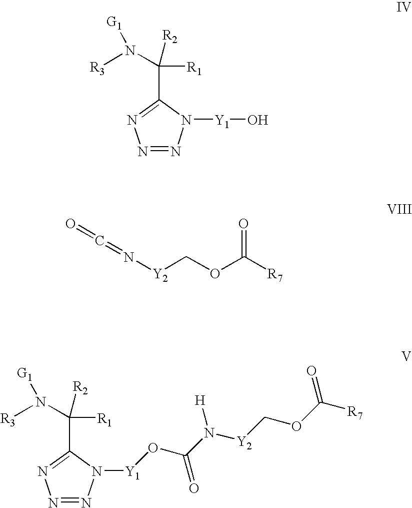
(b) deprotecting the compound of formula V to form a compound of formula VI or a pharmaceutically acceptable salt thereof; 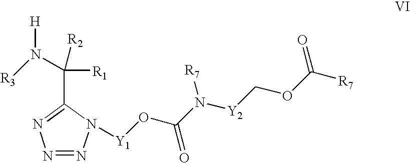
(c) reacting a compound of formula VI or a pharmaceutically acceptable salt thereof with a compound of formula XI, in the presence of a peptide coupling reagent to give a compound of formula VII; and 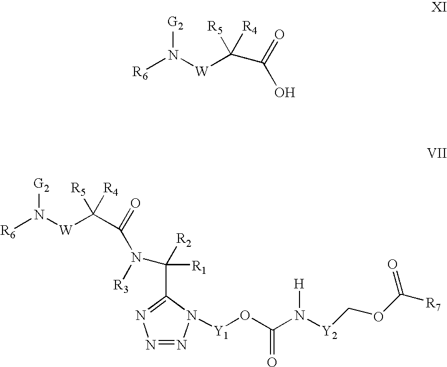
(d) deprotecting the compound of formula VII.

12. The process of claim 11, wherein G₁ is an amine protecting group selected from the group consisting of tert-butyloxycarbonyl, benzyloxycarbonyl and benzyl.

13. A compound having the structure:

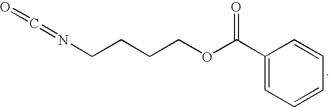

14. A compound having the structure:

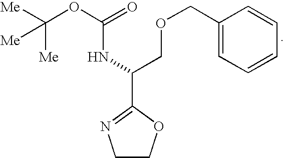

15. A compound having the structure:

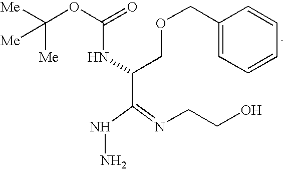

16. A compound having the structure:

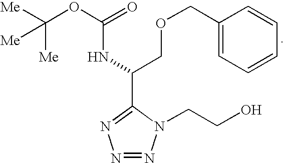

17. A compound having the structure: [chemical structure]
